(19) United States
(12) Patent Application Publication
(10) Pub. No.: US 2005/0032084 A1
(43) Pub. Date: Feb. 10, 2005

(54) Method of attaching a biopolymer to a solid support

(75) Inventors: Michael C. Pirrung, Mesonite, TX (US); Amy Odenbaugh, Morrisville, NC (US); Richard Connors, Pacifica, CA (US); Janice Worden, Rougemont, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC (US)

(21) Appl. No.: 10/752,493

(22) Filed: Jan. 8, 2004

Related U.S. Application Data
(63) Continuation of application No. 09/871,691, filed on Jun. 4, 2001, now abandoned.
(60) Provisional application No. 60/208,493, filed on Jun. 2, 2000.

(51) Int. Cl. B05D 3/00; C12Q 1/68; C12M 1/34

(52) U.S. Cl. 435/6; 435/287.2; 427/2.11

(57) ABSTRACT

The present invention relates, in general, to a method of attaching a biopolymer to a solid support and, in particular, to a method of attaching a nucleic acid to a glass surface, and to reagents suitable for use in such a method. The invention further relates to the product produced by the present method and to kits comprising same.

This application claims priority from Provisional Application No. 60/208,493, filed Jun. 2, 2000, the entire contents of which are incorporated herein by reference.

### TECHNICAL FIELD

The present invention relates, in general, to a method of attaching a biopolymer to a solid support and, in particular, to a method of attaching a nucleic acid to a glass surface, and to reagents suitable for use in such a method. The invention further relates to the product produced by the present method and to kits comprising same.

### BACKGROUND

The use of microarrays of nucleic acids (“DNA chips”) is revolutionizing many aspects of genetic analysis (Pease et al, Proc. Natl. Acad. Sci. USA 91:5022-5026 (1994), Woychik et al, Mutat. Res. 400:3-14 (1998), Lockhart et al, Nucleic Acids Symp. Ser. 38:11-12 (1998), Ramsay, Nat. Biotechnol. 16:40-44 (1998), Marshall et al, Nat. Biotechnol. 16:27-31 (1998), Nollau et al, Clin. Chem. 43:1114-1128 (1997), Southern, Trends Genet. 12:110-115 (1996)). While there are a number of methods to fabricate these arrays, including physical delivery through probes (Schena et al, Science 270:467-470 (1995), needles, channels (Maskos et al, Nucleic Acids Res. 20:1679-1684 (1992)), or jets (Stimpson et al, Proc. Natl. Acad. Sci. USA 92:6379-6384 (1995), as well as in situ synthesis (Matson et al, Anal. Biochem. 217:306-310 (1994)), including photolithography (Fodor et al, Science 251:767 (1991)), an underlying technology on which all DNA chips depend is the covalent attachment of oligonucleotides to flat surfaces, usually glass.

Classical methods of functionalizing glass through siloxane linkages generally have been used. A number of groups have reported various protocols for preparing glass microscope slides, a range of silicon-based attachment reagents, a diversity of linker and spacer groups, and various chemistries for attachment of the oligonucleotide (Guo et al, Nucl. Acids Res. 22:5456-5465 (1994), Joos et al, Anal. Biochem. 247:96-101 (1997), Chrisey et al, Nucleic Acids Res. 24:3031-3039 (1996), Henke et al, Anal. Chim. Acta 344:201-213 (1997), Yang et al, Chem. Lett. 257-258 (1998)). These methodologies are not ideal for use in the fabrication of arrays of oligonucleotides specifically presenting free 3′-ends for enzymatic processing (Shumaker et al, Hum. Mutat. 7:346-354 (1996), Shumaker et al, Ed: Landegren, U. Oxford University Press, Oxford, UK, Lab. Protoc. Mutat. Detect. pgs. 93-95 (1996)).

A method currently under development for genetic analysis involves the DNA polymerase-based solid-phase extension of primer-template complexes with labeled terminators, called APEX (Arrayed Primer Extension; FIG. 1) (Nikiforov et al, Nucl. Acids. Res. 22:4167-4175 (1994)). Unlike earlier processes of this type that attach the template to the support (Syvanen et al, Genomics 12:590-595 (1992), Syvanen et al, Genomics 8:684-692 (1990), Syvanen et al, Hum. Mutat. 3:172-179 (1994)), in APEX the primer is attached to the support. As the polymerase operates on the 3′-end of the primer strand of the primer-template complex, this necessitates that the primer be attached via its 5′ end. A reasonable approach to this goal is to incorporate linking units such as amino into the automated synthesis of primers using conventional machine methods. Such oligonucleotides have been used in spotting reactions on glass surfaces functionalized with epoxysilanes (FIG. 2). These are widely used, commercially available compounds, such as (glycidoxypropyl)triethoxysilane, which form a polymeric siloxane network that chemisorbs to silanol groups of glass (Plueddemann, Silane Coupling Agents, Plenum, N.Y. (1991)). The results of DNA immobilization on such surfaces can be inconsistent. Epoxide functional groups remain that could potentially react with nucleophiles (such as proteins) in subsequent analysis steps. Further, these surfaces exhibit a range in shelf life. Some of these behaviors could be explained by the formation of a multilayer siloxane structure, rather than the monolayer idealized in many reports. Another disadvantage of epoxy derivatization protocols is that spotting of amino-derivatized oligonucleotides must be conducted in strongly alkaline solutions so that the amine is present in its free base form. This leads to metal hydroxide precipitation in the spots during evaporation, which may result in degradation of the siloxane attachment. Such basic oligonucleotide solutions are also quite corrosive, raising issues with the long term capability to microfabricate DNA arrays using metallic components.

A very substantial literature exists on the functionalization of solid supports for chromatographic separations. For example, in the functionalization of polysaccharide supports with affinity chromatography ligands, use is made of the alkylation of oligonucleotides derivatized with a phosphorothioate at their 5′-ends as reported by Letsinger (Gryaznov et al, J. Am. Chem. Soc. 115:3808-3809 (1993), (Herrlein et al, Nucl. Acid Res. 22:5076-5078 (1994)) with bromoacetyl-functionalized agarose (Kang et al, Nucl. Acids Res. 23:2344-2345 (1995)). The required 5′-phosphorothioate DNA can be prepared synthetically or by treatment of oligonucleotides with T4 kinase and γ-thio-ATP. In the derivatization of silica gel, monovalent silanes have been observed to produce more uniform and reproducible derivatization than polyvalent silanes (Pesek et al Eds. Chemically Modified Surfaces: Recent Developments, The Royal Society of Chemistry, Cambridge, UK (1996), Wheatley et al, Chromatography 726:77-90 (1996)). At the same time, a potential drawback of monovalent derivatizing reagents is that the stability of their attachment to the surface may be reduced compared to polymeric networks.

The method of the present invention is based on a new chemistry for attaching oligonucleotides to glass surfaces via their 5′-ends. The present bromoacetyl/phosphorothioate method offers the advantage of rapid reaction under mild conditions.

### SUMMARY OF THE INVENTION

The present invention relates to a method for attaching a biopolymer (e.g., a nucleic acid) to a solid support (e.g., glass) using silanes and bromoacetamide/phosphorothioate linking chemistry. The invention further relates to specific bromoacetamide silanes suitable for use in support derivatization. The invention additionally relates to support-bound biopolymers produced in accordance with the present method.

### BRIEF DESCRIPTION OF THE DRAWINGS

FIG. 1. The APEX reaction: Extension of a surface bound primer strand occurs by hybridization to a template strand in solution, recognition of this primer-template complex by a DNA polymerase, and the addition of a labeled terminating nucleotide triphosphate. When primers are arrayed on the surface, the method permits parallel analysis of many single-nucleotide sites in analyte DNA.

FIG. 2. Conventional chemistry for immobilization of amine-derivatized oligonucleotides on epoxysilane-functionalized surfaces.

FIG. 3. Confocal fluorescence micrograph of oligonucleotide 3 spotted on a slide functionalized with silane A and subjected to APEX with template 7. The average fluorescence intensity across the spot is 88, while the average fluorescence intensity in the nearby dark region is 3.5. The mean diameter of the spot is 200 μm.

FIG. 4. APEX reactions were conducted on arrays formed from oligonucleotides 2, 3, 4, and 5 with templates 6, 7, 8, and 9. Spot sizes are ˜180 μm.

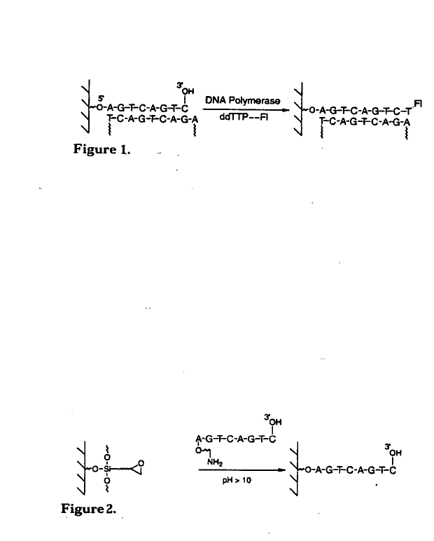

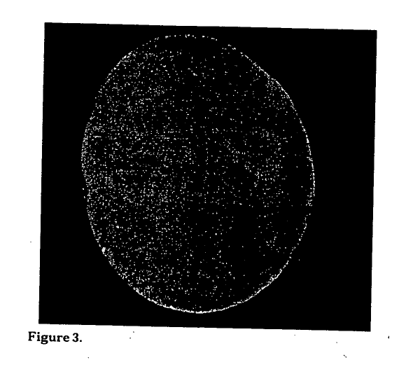

### DETAILED DESCRIPTION OF THE INVENTION

The present invention relates to bromoacetamidosilanes and to the use thereof for modifying (functionalizing) solid supports. The invention further relates to supports modified with bromoacetamidosilanes and to use of same in the preparation of arrays of biopolymers.

Bromoacetamidosilanes suitable for use in the invention are of Formula I: 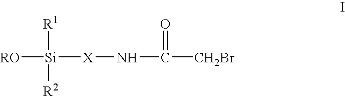
wherein:
R is an aryl or linear or branched alkyl. Preferably, R is a linear or branched C₁-C₁₀ alkyl. More preferably, R is ethyl or methyl;
R¹ and R² are, independently, a linear or branched alkyl, linear or branched alkoxy, aryl or phenoxy. Preferably, R¹ is a C₁-C₁₀ linear or branched alkyl and R² is a C₁-C₁₀ linear or branched alkyl, a C₁-C₁₀ linear or branched alkoxy or phenoxy. More preferably, R¹ is a C₁-C₅ linear or branched alkyl and R₂ is a C₁-C₁₀ linear or branched alkyl or a C₁-C₁₀ linear or branched alkoxy. Most preferably, R¹ is methyl, ethyl, propyl or isopropyl and R² is a C₁-C₅ linear or branched alkyl or methoxy or ethoxy; and
X is a linker group, for example, (CH₂)ₙ wherein n is 0 to 25, preferably 2 to 10, more preferably 3 or 4, an ethylene glycol oligomer or an aryl group.

Specific silanes for use in the invention include N-(3-diethoxymethylsilylpropyl)bromoacetamide (DiOEt), N-(3-dimethyl-ethoxysilylpropyl)bromoacetamide (DiMe) and N-(4-disopropylmethoxylsilylbutyl)bromoacetamide (DiIso):

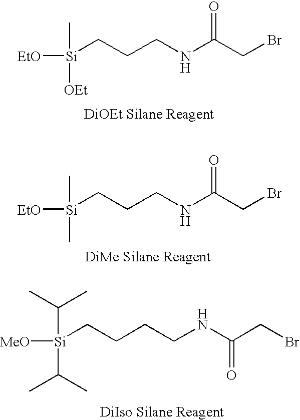

Silanes can be selected for optimum signal to noise (S/N) ratios in the context used. For example, DiMe silanized glass supports have high and consistent S/N ratios of the fluorescence of fluorescein-ddATP tagged APEX spots versus background fluorescence. As both monoalkoxy and dialkoxy silanes produce monolayered films on glass surfaces (as opposed to trialkoxy silanes, which can form complex layers of coating on surfaces), the differences in the APEX reactivities between DiMe, DiOEt and DiIso glass supports may lie in the differences in the R¹ and R² groups. While not wishing to be bound by theory, it is believed that, in the context of APEX, differences in these groups (R¹ and R²) affect the density of bromoacetamide groups on a glass support surface and, therefore, the amount of space available for hybridization between solution-phase oligonucleotides and the tethered oligonucleotides. R¹ and R² can also affect the hydrophobicity or hydrophilicity of the array surface, which may affect the reactivity of the DNA polymerase at the hybrid sites. For example, very hydrophobic surfaces may make bringing polymerase close to the surface of the array energetically costly. Excessively hydrophilic surfaces, on the other hand, may cause difficulty in creating small arrays of large numbers of different pre-synthesized oligonucleotides, as aqueous oligonucleotide solutions tend to spread out extensively when placed on hydrophilic surfaces during the creation of the arrays. A balance of these surface-modification factors results in a oligonucleotide coupling reagent of the preferred reactivity. By way of example, oligonucleotide densities for glass supports coated with the above-referenced reagents are as follows: 0.15×10⁻¹⁰ mol/mm² for DiMe, 0.12×10⁻¹⁰ mol/mm² for DiOEt and 0.05×10⁻¹⁰ mol/mm² for DiIso.

Supports suitable for derivatization using the compound of Formula I include glass or silicon supports, for example, glass or silicon chips (or microchips) or oxidized (SiO₂) silicon wafers, such supports being appropriate for use in a variety DNA analytical techniques, including APEX. As used herein, a “chip” is a small flat surface to which a multiplicity of molecules can be attached to a multiplicity of locations. A microchip is such a surface wherein the dimensions of each location can be in the range of 50 to 1000, preferably, in the range of 100 to 500 microns, and most preferably in the range of 200 to 300. Advantageously, the support is cleaned prior to treatment. In the case of glass supports, cleaning can be effected by immersion in a strong base (e.g., 1 M KOH/Decon solution) at an elevated temperature (e.g., 60° C.) followed by multiple rinses in deionized water and then immersion in a strong acid (e.g., 1M HCl). The support can then be further rinsed with water and dried. The support can then be treated with the bromoacetamidosilane using any convenient method, a thin-film method and a solution coating method are described in the Examples that follow.

Biopolymers that can be affixed to the derivatized support include nucleic acids (DNA or RNA), proteins or peptides or carbohydrates. Depending on the ultimate use of the immobilized biopolymer, it can bear a detectable (e.g., fluorescent) label, or it can be unlabeled.

In the case of DNA, use of a 5′ phosphorothioate DNA is preferred particularly when the immobilized DNA is to be used in APEX reactions (see Examples that follow). A nucleophilic reaction of the phosphorothioate with the bromoacetyl group results in the covalent linkage of the DNA to the bromoacetylsilane-derivatized glass surface. Optimum conditions for the reaction can be readily determined by one skilled in the art, however, examples of suitable conditions are provided in the Examples below. It will be appreciated from a review of the Examples that the phosphothioate/bromoacetamide linking reaction can be carried out under mild conditions. The resulting siloxane linkages are stable to both the aqueous solvent and thermal conditions used in APEX. DNA immobilized in accordance with the present method can be used for purposes other than APEX, which requires a free 3′ end. When used for other purposes (e.g., hybridization), attachment of the nucleic acid can be via the 3′ or 5′ end.

The use of 5′-phosphorothioates is particularly advantageous since, in addition to being capable of being prepared by chemical synthesis, they can be generated by kinase reactions on native DNA. Therefore, the present approach is applicable not only to shorter, synthetic oligonucleotides but also to biologically derived, larger oligonucleotides. This linking chemistry also proceeds at relatively dilute phosphorothioate concentrations, thereby obviating a processing step in the use of natural nucleic acids.

After immobilization of the biopolymer (for example, DNA, although the present invention is applicable to essentially any phosphorothioate-bearing molecules), the support surface can be treated with a passivating reagent. Reagents that react with the electrophilic bromoacetamide groups but maintain the hydrophobicity of the surface are preferred. As an example, overnight treatment with thiosulfate (e.g., 0.25 M) can be used.

The support bound biopolymers prepared in accordance with the present method can be used in a variety of clinical (including diagnostic) and research settings. For example, DNA bound to glass chips can be used in APEX reactions. In APEX, an oligonucleotide array, tethered to a solid support (e.g., glass chip) is exposed to a solution of oligonucleotides under the proper hybridization conditions. The oligonucleotide solution also contains a DNA polymerase and labeled dideoxynucleotide triphosphates. The polymerase tags the oligonucleotide hybrids formed between the tethered and solution-phase oligonucleotides with the labeled dideoxynucleotides. The array is then rinsed to remove unhybridized oligonucleotides and any unused, labeled nucleotides. Through the use of this method, a 10-mer DNA chip can sequence a cosmid in about one hour, a throughput of 1 Kb/min. Such DNA chips with oligonucleotide arrays designed to probe for a specific gene like the p53 gene, important as an indicator of many tumors, can be manufactured in bulk and used in hospitals, clinics and doctor's offices to quickly ascertain a patient's likelihood of a genetically-linked conditions. Other DNA chips with suitable oligonucleotide arrays provide useful tools in other areas requiring DNA sequence information, such as forensic chemistry or population genetics.

The derivatized support of the invention can be provided as kits comprising the support disposed within a container means. The derivatized support can be present in the kit bound to a biopolymer or free of biopolymer.

Certain aspects of the present invention will be described in greater detail in the Examples that follow. The entire contents of the following publications are incorporated herein by reference: Pirrung et al, Langmuir 16:2185 (2000), Pirrung et al, J. Am. Chem. Soc. 122:1873 (2000).

### EXAMPLES

The following experimental details are relevant to the specific Examples that follow.

General. Spectroscopy. All ¹H NMR and ¹³C NMR spectra were recorded on a Varian INOVA 400 spectrometer. Microscopy. Confocal laser-scanning epifluorescence microscopy was performed with a BioRad MRC-1000, Zeiss Axioscope (10× objective), and a Kr/Ar laser (Model 5470K-00C-2B, Ion Laser Technology, Salt Lake City, Utah) at an excitation wavelength of 488 nm. Quantitative signal-to-noise ratios (S/N) were obtained in the photon counting mode under a specified laser intensity (% L), iris aperture (I), gain (G) [most images were obtained with a multiplier of 16], and scan speed (SS) by measuring the average pixel intensity of the slide background (areas that had not been exposed to any oligonucleotide) and a representative and/or large area of each spot using software supplied with the microscope. Thermal cycling was performed on a Perkin Elmer 480 Thermal Cycler.

Reagents and Supplies. (3-Aminopropyl)methyldiethoxysilane was from Fluka. (3-Aminopropyl)dimethylethoxysilane and (3-cyanopropyl)diisopropylmethoxy chlorosilane were from Gelest or UCT. Bromoacetyl bromide was from Aldrich. Spacer Phosphoramidite 18, Phosphorylating Agent II, 3H-1,2-benzodithiole-3-one-1,1-dioxide (Beaucage reagenti), fluorescein-CPG, Poly-Pak cartridges, and Poly-Pak II cartridges were from Glen Research (Sterling, Va.). PCR buffer, ddCTP, ddGTP, dTTP, and Amplitaq Polymerase were from Perkin Elmer. Fluorescein-ddATP was from NEN.

N-(3-diethoxymethylsilylpropyl)bromoacetamide (A). A 1 L round bottom flask was charged with 500 mL of dry ether, fitted with a dropping funnel, flushed with nitrogen, and placed in a dry ice/acetone bath. Bromoacetyl bromide (13.5 mL, 31.3 g, 155 mmol) was added. Triethylamine (21.6 mL, 15.7 g, 155 mmol), freshly distilled from CaH₂, was added slowly, which caused formation of a white precipitate. The dropping funnel was rinsed with 10 mL of dry ether and charged with a solution of 29.7 g of (3-aminopropyl)methyldiethoxysilane (32.5 mL, 155 mmol) in 160 mL of ether. After 5 min, the silane solution was added dropwise to the reaction mixture. The reaction was allowed to stir for 2 h at −78° C. and was vacuum filtered through a layer of Celite 545 in a medium fritted-glass filter. The yellow filtrate was concentrated under reduced pressure and purified by flash chromatography (silica gel, eluent=2:1 hexanes:ethyl acetate) to give 32.0 g (66% yield) of a yellow oil. ¹HNMR (CDCl₃): δ 0.10 (m, 3H); 0.58 (m, 2H); 1.19 (m, 6H); 1.58 (m, 2H); 3.24 (m, 2H); 3.72 (m, 4H); 3.83 (s, 2H); 6.69 (br s, 1H). ¹³CNMR (CDCl₃): 6-5.0, 11.1, 18.3, 22.7, 29.3, 42.6, 58.2, 165.2. HRMS (FAB⁺): m/z (M−H)⁺ calculated for C₁₀H₂₃BrNO₃Si, 312.0834, found 312.0645.

N-(3-dimethylethoxysilylpropyl)bromoacetamide (B). This compound was synthesized as above except that 3-aminopropyldimethylethoxysilane was used in place of the 3-aminopropyldiethoxymethylsilane. Purification again was via flash chromatography (silica gel, eluent=2:1 hexanes:ethyl acetate), producing a golden yellow oil in 74% yield. ¹H NMR (CDCl₃): δ 0.03 (m, 6H); 0.51 (m, 2H); 1.10 (t, J=7.0 Hz, 3H); 1.49 (m, 2H); 3.18 (m, 2H); 3.58 (m, 2H); 3.78 (s, 2H); 6.79 (br s, 1H). ¹³CNMR (CDCl₃): δ −2.3, 13.4, 18.3, 23.0, 29.1, 42.7, 58.1, 165.4. HRMS (FAB⁺): m/z (MH)⁺ calculated for C₉H₂₁BrNO₂Si, 284.1538, 284.0498.

(3-Cyanopropyl)diisopropylmethoxysilane. (3-cyanopropyl)diisopropyl chlorosilane (10.3 mL, 10.0 g, 45.9 mmol) was placed in a 50 mL round bottom flask fitted with an addition funnel, kept under an argon atmosphere, and chilled in an ice bath. Pyridine (6.31 mL, 6.17 g, 78.0 mmol, freshly distilled from CaH₂) was added. Methanol (5.39 mL, 4.26 g, 133 mmol, freshly distilled from NaOMe) was added dropwise to the reaction mixture. The mixture was allowed to stir overnight at room temperature. The clear solution was washed with water, 5% H₂SO₄, and 10% NaHCO₃. The organic layer was dried over Na₂SO₄ and filtered to give 9.25 g (94%) of a clear liquid. ¹H NMR (CDCl₃): δ 0.75 (m, 2H); 0.99 (m, 14H); 1.73 (m, 2H); 2.35 (t, J=6.8 Hz, 2H); 3.47 (s, 3H). ¹³C NMR (CDCl₃): δ 9.9, 12.2, 17.4, 20.2, 20.8, 53.9, 119.8.

(4-Aminobutyl)diisopropylmethoxysilane. A 1M suspension of LiAlH₄ (31.9 mL dry diethyl ether, 1.21 g LiAlH₄, 31.9 mmol) was prepared in a three-necked flask. The mixture was placed under argon pressure and chilled in an ice bath. 3-cyanopropyldiisopropylmethoxysilane (3.10 g, 2.2 mL, 14.5 mmol) was dissolved in 35 mL dry ether and added dropwise to the suspension. After completion of the nitrile addition, the reaction mixture was removed from the ice bath and stirred for 45 min. The reaction mixture was quenched by slow, successive additions of 2 mL H₂O, 2 mL saturated Na₂CO₃, and 5 mL H₂O. The ether layer was washed with saturated Na₂CO₃, dried over Na₂SO₄, and concentrated under reduced pressure to leave a white, cloudy oil, which was purified via Kugelrohr distillation (55-60° C., 5 torr) to yield 1.35 g of clear liquid (47%). ¹HNMR (CDCl₃): δ 0.58 (m, 2H); 0.96 (m, 14H); 1.39 (m, 6H); 2.62 (m, 2H); 3.42 (s, 3H). ¹³CNMR (CDCl₃): δ 10.1, 12.2, 17.5, 20.6, 37.9, 41.7, 51.3.

N-(4-diisopropylmethoxysilylbutyl)bromoacetamide (C). This compound was synthesized as for A above, excepting that (4-aminobutyl)diisopropylmethoxysilane was used in place of the (3-aminopropyl)diethoxymethylsilane and the reaction was allowed to proceed for 1 h only at −78° C. prior to the filtration step. Purification via flash chromatography (silica gel, eluent=1:1 hexanes:ethyl acetate) produced a yellow oil in 66% yield. ¹HNMR (CDCl₃): δ 0.61 (m, 2H); 0.94 (m, 14H); 1.38 (m, 2H); 1.43-1.57 (m, 2H); 3.42 (s, 3H); 3.81 (s, 2H); 6.57 (br s, 1H). ¹³CNMR (CDCl₃): δ 10.9, 13.3, 18.6, 21.7, 30.3, 34.1, 40.7, 52.4, 166.4. HRMS (FAB+): m/z (M⁺−H) calculated for C₁₃H₂₇BrNO₂Si, 336.1194, found 336.0992.

Slide Cleaning. Microscope slides (3″×1″×1 mm, Propper Select) were cleaned ten at a time by being placed in a glass rack and immersed in an aqueous 1M KOH/1% Decon solution (500 mL) at 60° C. for 30 min. The slides were rinsed in five successive deionized water baths (250 mL each). The slides were then placed in an ethanolic 1M HCl bath (500 mL) at room temperature for 30 min. The slides were rinsed in another five water baths and dried for 16-78 h in a 60° C. oven. After cleaning, the slides were stored in a desiccator until ready for use.

“Thin-film” Silanization. Silane (9 μL) was pipetted lengthwise in a line on the face of a microscope slide. A cover slip (24×50 mm, VWR #48393081) was placed over the slide and pressed down with the back of a pair of tweezers to spread the silane to cover the entire slide surface. Slides were placed in a rack, covered with aluminum foil or a box to prevent dust contamination, and left with the cover slips applied for 1 h at room temperature. The cover slips were removed and the slides were rinsed thoroughly with a stream of absolute ethanol. The slides were allowed to air dry, transferred to a 120° C. oven for 2 h, and cooled in a desiccator. Slides not used immediately were stacked, wrapped in plastic wrap, and kept in a desiccator.

Solution Silanization. Ethanol or acetone solutions containing 2%-8% silane (by volume) and 5% water were prepared. A few drops of 5% H₂SO₄ were added to the solution to lower the pH to about 6. Clean slides were immersed in the solutions for 3 min (three slides in 35 mL of solution), removed and immediately washed in four successive acetone baths (250 mL each). The slides were allowed to dry only after removal from the fourth acetone bath. The slides were cured in an oven at 120° C. for 165 min.

DNA Synthesis. Oligodeoxyribonucleotides were synthesized using solid-phase phosphoramidite methodology on an Applied Biosystems 392 DNA/RNA automated synthesizer. Table 5 lists the oligonucleotide sequences, all of which include a 5′-spacer 18 at the penultimate 5′-position and which are terminated with a 5′-phosphorothioate. These oligonucleotides were synthesized using normal synthesis cycles for the conventional nucleoside phosphoramidites and the Spacer Phosphoramidite 18. As recommended in a protocol provided by Glen Research, generation of the phosphorothioate was accomplished by replacement of the bottles containing Spacer Phosphoramidite 18 and oxidizing reagent with Phosphorylating Agent II and Sulfurizing Reagent (3H-1,2-benzodithiole-3-one-1,1-dioxide). Some of the 5′-phosphorothioate oligonucleotides also include fluorescein at the 3′-terminus, which were synthesized using fluorescein-CPG. The oligonucleotides were purified via Poly-Pak or Poly-Pak II cartridges. After the purified oligonucleotides were dried, the yield was determined by dissolving a known percentage of the oligonucleotide in deionized water and measuring the absorbance of the sample at 260 nm. (An absorbance of 1.0 equals a concentration of 20 μg/mL of oligonucleotide.)

TABLE 1

Oligonucleotide sequences synthesized in this
study, listed from 5′ to 3′

| | |
|---|---|
| Oligonucleotide | |
| Number | Sequence |

| | |
|---|---|
| 1 | JSd(CG CGA GGT CGC ACG GCT CAG)F |
| 2 | JSd(CG CGA GGT CGC ACG GCT CAG AAA |
| | AA) |
| 3 | JSd(CG CGA GGT CGC ACG GCT CAG AAA |
| | AT) |
| 4 | JSd(CG CGA GGT CGC ACG GCT CAG AAA |
| | AG) |
| 5 | JSd(CG CGA GCT CGC ACG GCT CAG AAA |
| | AC) |
| 6 | d(TTT TTT TTT CTG AGC CGT GCG ACC |
| | TCG CG) |
| 7 | d(TTT TAT TTT CTG AGC CGT GCG ACC |
| | TCG CG) |
| 8 | d(TTT TCT TTT CTG AGC CGT GCG ACC |
| | TCG CG) |
| 9 | d(TTT TGT TTT CTG AGC CGT GCG ACC |
| | TCG CG) |
| 10 | JSd(CG CGA GGT CGC ACG GCT CAG AAA |
| | TA) |
| 11 | JSd(CG CGA GGT CGC ACG GCT CAG AAA |
| | TT) |
| 12 | JSd(CG CGA GGT CGC ACG GCT CAG AAA |
| | TG) |
| 13 | JSd(CG CGA GGT CGC ACG GCT CAG AAA |
| | TC) |
| 14 | d(TTT TTA TTT CTG AGC CGT GCG ACC |
| | TCG CG) |
| 15 | d(TTT TAA TTT CTG AGC CGT GCG ACC |
| | TCG CG) |
| 16 | d(TTT TCA TTT CTG AGC CGT GCG ACC |
| | TCG CG) |
| 17 | d(TTT TGA TTT CTG AGC CGT GCG ACC |
| | TCG CG) |

F denotes fluorescein,
J denotes phosphorothioate [S—PO₂—O—],
S denotes Spacer-18, [—(CH₂CH₂O)₆—PO₃—].

Oligonucleotide Spotting. 5′-Phosphorothioate oligonucleotides were dissolved in deionized water at 1 mM-0.125 mM concentration. The oligonucleotide solutions were spotted onto silanized slides in 0.4-0.2 μL aliquots from a 10 μL Pipetman pipette tip (RT-S10, Rainin). (This was essentially the smallest drop that was possible using these tips). These spots were approximately 2 mm in diameter. Smaller spots could be made using elongated glass micropipettes (approximately 0.30-0.18 mm in diameter). The spotted slides were placed in 50 mL plastic tubes containing 1.0-0.2 mL of deionized water to keep the chamber humid, thereby keeping the oligonucleotide spots from drying. The slides were removed from their humid chambers after 1 h and rinsed thoroughly under a stream of deionized water. This incubation time is likely extremely conservative. Preliminary experiments indicated that 15-30 min incubations did not result in significantly different oligonucleotide attachment. The slides were placed in a rack and allowed to air dry in a dark, dust free container. Slides spotted with fluorescently tagged oligonuclotides could be visualized by fluorescence microscopy (30% laser power, 10× objective lens, scan speed=slow, Gain=1500, Iris=6.0, photon counting mode). Immediately prior to visualizing each slide, 24 μL of pH 10 buffer (Fisher) was pipetted onto the slide and a cover slip was applied.

APEX Reactions. Slides spotted with the non-fluorescently tagged 5′-phosphorothioate oligonucleotide 3 (see Table 1) were fitted with a 50 μL cover well (Aldrich-Sigma). An APEX reaction mix (100 μL) containing the following [15.0 μL 10×PCR buffer; 0.4 μL ddCTP (10 mM); 0.4 μL ddGTP (10 mM); 0.4 μL dTTP (10 mM); 1.0 μL Fluorescein-ddATP (1 mM); 2.0 μL Amplitaq Polymerase (5 U/μL); 15.0 μL 25 MM MgCl₂; 12.5 μL complementary oligonucleotide 7 (see Table 1) (0.165 mM); 103.8 μL deionized water] was placed between the cover well and the slide using a 1 mL tuberculin syringe. The slide was then subjected to 20 thermal cycles. One cycle consisted of heating the slides to 90° C. for 1 min, cooling to 37° C. for 1 min, heating to 70° C. for 2 min. At the end of twenty cycles, the slides were held at 0° C. for 1 h to up to 20 h. The cover well was removed and the APEX reaction mix was thoroughly rinsed from the slide under a stream of deionized water. After the slide was allowed to air dry, 24 μL of pH 10 buffer was placed on the slide and a cover slip was applied. S/N ratios of the fluorescence of the fluorescein-ddATP tagged spots were determined as before, using the confocal microscope. Signal to noise (S/N) of smaller APEX spots made by spotting with elongated micropipettes averaged approximately eight times higher than APEX spots made with plastic pipette tips. Initial shelf-life studies indicate that the silanized slides, when kept in a desiccator, are able to retain high reactivity for 5′ thiophosphate oligonucleotides for 3-4 months.

### Example 1

### Synthesis of the Bromoacetamidosilanes, and Slide Derivatization

Three novel bromoacetamido silanes were prepared by acylation of the corresponding amines as described in the above. For A and B (defined above and below), this precursor is commercially available. For C (defined above and below), it was prepared from the corresponding (methoxydiisopropylsilyl)butyronitrile by LiAlH₄ reduction.

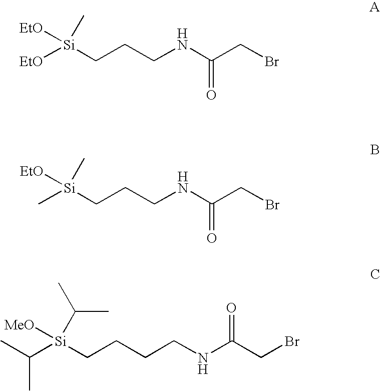

Functionalization of glass microscope slides with these silanes was conducted by two methods. Thin film derivatizations were performed in which neat silane was placed along one edge of the slide and a cover slip was lowered onto it to spread the silane over its surface. This method has the virtue that it consumes small amounts of silanes, though it is less amenable to scale-up. For larger-scale experiments, solution-phase derivatizations were performed by dipping slides into 2-8% solutions of silane in ethanol or acetone. Regardless of the method of silane application, slides were subsequently oven-cured. Observations of the resulting slides were instructive as to the success of the functionalization. In general, unfunctionalized slides are hydrophilic, as evidenced by spreading of water on the surface. Well functionalized slides are quite hydrophobic, as evidenced by beading of water on the surface. When DNA is successfully immobilized, the spots become hydrophilic and in most cases are readily visualized.

### Example 2

### Fluorescent DNA Immobilization

Initial investigations of the ability of slides coated as described in Example 1 to immobilize DNA by nucleophilic reactions with the bromoacetyl groups used a 5′-phosphorothioate DNA bearing a 3′-fluorescein (1):

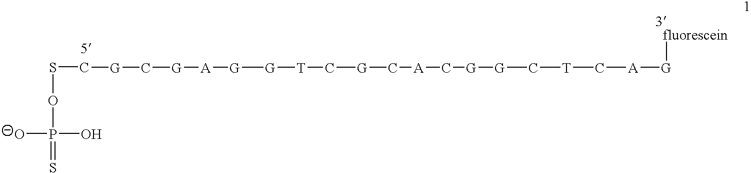

To compare with other 5′ chemistries, analogous fluoresceinated oligonucleotides with 5′-phosphate and 5′-aminolink groups were also used. Data for the average S/N for octuplicate spots are summarized in Table 2. Bright spots were seen for each of the 5′ linking chemistries, but particularly so for the phosphorothioates. The aminolink and phosphate oligonucleotides did not survive APEX conditions, however, implying they were not covalently attached (vide infra).

TABLE 2
Average signal-to-noise ratio of fluorescent DNA spotted onto
bromoacetylsilane-derivatized glass slides.

| Pre | A | B | C |
|---|---|---|---|
| Phosphorothioate | 50 | 68 | 35 |
| Aminolink | 14 | 12 | 13 |
| Phosphate | 19 | 48 | 23 |

The influence of the spotting solution on immobilization was examined. Earlier work on the bromoacetyl/phosphorothioate reaction suggested that pH 8 phosphate buffer would provide the largest reaction rate. Spots made in water were brighter, as shown in Table 3, but those made in buffer tended to be more uniform. Because of its simplicity, water was adopted as the spotting solvent.

TABLE 3
Average signal-to-noise ratio of fluorescent
phosphorothioate DNA spotted in water or phosphate
buffer onto bromoacetylsilane-derivatized glass
slides the indicated number of days after coating.

| phosphate | A | B | C | H₂O | A | B | C |
|---|---|---|---|---|---|---|---|
| 0 | 26 | 97 | 16 | | 33 | 106 | 27 |
| 1 | 44 | 47 | 35 | | 62 | 64 | 53 |
| 2 | 43 | 35 | 32 | | 54 | 51 | 41 |

The stability of A-, B-, or C-derivatized slides over time was examined. In a short-term study, little loss of immobilization activity was observed within two days after coating. Spotting three concentrations of 1 (in quadruplicate) at one and seventeen days after coating and detection of immobilized fluorescence after washing as summarized in Table 4 reveals no experimentally significant differences.

TABLE 4
Average signal-to-noise ratio of fluorescent DNA
spotted at the indicated concentration onto
bromoacetylsilane-derivatized glass slides 1
and 17 days after silane coating.

| 1 day | A | B | C | 17 days | A | B | C |
|---|---|---|---|---|---|---|---|
| 2 mM | 49 | 59 | 17 | | 60 | 71 | 12 |
| 1 mM | 51 | 65 | 19 | | 62 | 73 | 16 |
| 0.1 mM | 57 | 64 | 26 | | 63 | 73 | 17 |

The effect of DNA concentration on immobilization was studied in the range 0.5 mM to 0.01 mM (with silane A) and 0.5 mM to 0.001 mM (with silane C). In general, the intensity of the fluorescence of DNA spots with this chemistry far exceeds earlier observations with epoxy chemistries, easily saturating the detector after one scan using conventional microscope parameters at a DNA concentration ≧250 μM. In further experiments with A, S/N as large as 150:1 were observed, and with C, as large as 120:1.

Phenomenological observations of these silane surfaces include the ease of making small spots due to high surface tension. The rate of reaction to immobilize DNA to bromoacetylsilane surfaces is believed to be very rapid. No increase in immobilization from 15 to 30 min was seen, and in experiments conducted in the presence of an air current, only a small amount of streaking of the immobilized DNA was observed even though the spotting solution was driven across the slide. This suggests that all of the DNA had been consumed before the spot could evaporate/move.

### Example 3

### Primer Extension Reactions

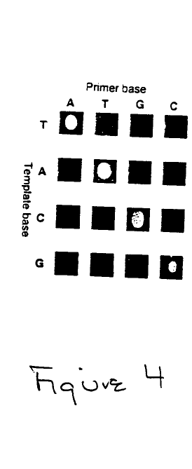

APEX reactions on A-, B-, and C-functionalized surfaces (see Examples 1 and 2) were examined with phosphorothioate oligonucleotide 3 and the complementary template 7 (see Table 1). The reaction mix includes AmpliTaq polymerase, fluoresceinated ddATP, and the non-complementary terminators, is sealed onto the face of the slide with a coverwell, and is subject to thermal cycling. One spot from an A slide is shown in FIG. 3. It reflects the high S/N observed in APEX reactions with phosphorothioate attachment chemistry, averaging 23:1 and comparing favorably to the signal observed when fluoresceinated DNA is immobilized. Study of the dependence of APEX on the concentration of DNA spotted onto derivatized slides shows that, for A and B, the DNA concentration should exceed 0.05 mM, while for C, the DNA concentration should exceed 0.005 mM. Only a modest ˜two-fold increase in APEX signals was observed when the concentration of the oligonucleotide spotting solution was increased four-fold, suggesting the spotted area was close to being saturated with oligonucleotide. Direct comparison of A, B, and C surfaces in APEX with aminolink and phosphorothioate DNA showed that there is no APEX (and likely no covalent attachment) with aminolink under standard spotting conditions. This is understandable as the amine should be protonated and therefore unreactive at neutral pH. An aged A slide (2.5 mo old) was examined for its ability to immobilize 3 and undergo APEX; it was undiminished compared to control.

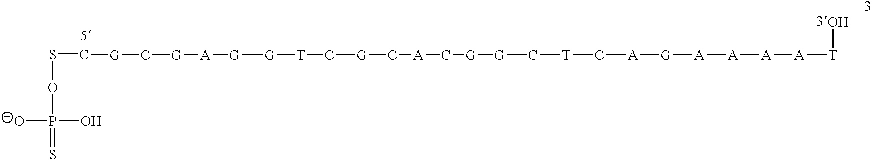

Data collected to this point had been from slides coated by the thin film method, so the use of slides prepared by alternative coating protocols in APEX was examined. A 1% solution of A in aqueous acetone produced slides that immobilized 1 with comparable S/N to the thin film method, so it was studied in APEX with 3. These results were also generally comparable to the thin film coating, and increasing the silane concentration to 4% did not lead to improvement. A systematic study of different coating methods was conducted with B. Table 5 lists the average signal measurements (S) of at least four spots in APEX reactions with the 3/7 pair on slides coated as shown. Standard deviations (SD) were calculated from each of these values to determine the consistency of reactivity with each type of slide. The size of the standard deviations is shown as the percentage of the average signal (% SD=SD/S×100). A film-coated slide was prepared, and two solution-coated slides were prepared from silane solutions aged at least 3 days. This aging process seems to lead to a higher concentration of hydrolyzed silane and aid slide coating. The concentrations listed are for the oligonucleotide 3 spotting solution. The data indicate that the ethanol solution-coated B slides produce spots that are not only more fluorescent, but have more spot-to-spot consistency.

TABLE 5
APEX signals as a function of the coating method with silane B.

| Coating method | 500 μM 3 S | % SD | 375 μM 3 S | % SD | 125 μM 3 S | % SD |
|---|---|---|---|---|---|---|
| film | 9.7 | 68 | 8.7 | 24 | 2.8 | 24 |
| 2% silane (acetone) | 9.6 | 35 | 9.9 | 20 | 2.8 | 15 |
| 2% silane (EtOH) | 26 | 13 | 19 | 9.0 | 3.5 | 23 |
| 4% silane (EtOH) | 19 | 20 | 17 | 12 | 3.6 | 20 |
| 8% silane (EtOH) | 22 | 20 | 23 | 11 | 2.3 | 34 |

It may be desirable to remove superfluous electrophilic sites from slides after the DNA is immobilized. The removal of reactive bromoacetyl sites was studied by spotting with 1 and imaging those spots, incubating with a passivating reagent, spotting additional 1 in new locations, and comparison of the whole slide to the pre-passivation image. The perfect passivating reagent would leave the original spots untouched, prevent the immobilization of additional 1, and not interfere with APEX. Reagents examined as passivators included acetate, adenosine monophosphate, adenosine monophosphorothioate, 2-(2-aminoethoxy)ethanol, aqueous ammonia (30%), azide, borate/carbonate buffer (pH 10), cyanide (0.5 M), morpholine, phosphate buffer (pH 7.8), phthalimide, pyridine, thiophosphate (250-375 mM), thiosulfate, and thiourea, for 1 h or overnight. Buffer (pH 10) was able to eliminate the reactivity of slides with new fluorescently tagged 5′ phosphorothioate oligonucleotides. Aqueous ammonia or cyanide reduced the reactivity of slides by 75%, and thiophosphate reduced their reactivity by 66%. Unfortunately, the best passivating agents also negatively affect the ability of oligonucleotides already tethered to undergo the APEX reaction. Overall, the preferred protocol, that still allows significant APEX, entails treatment of slides with thiosulfate (250-500 mM) for 16 h, which reduces the signal of 1 applied to bromoacetyl slides to 76% (A), 68% (B), and 54% (C) of the value before passivation. When slides derivatized with 3 and passivated in this way were subjected to APEX, the signal was decreased to 64% (A), 22% (B), and 39% (C). APEX of oligonucleotides spotted prior to passivation generally had S/N two to three times that of oligonucleotides spotted after treatment of the slide with thiosulfate. As APEX reactions on slides not subjected to passivation were still successful, further experiments omitted this treatment.

An example of the utility of APEX with bromoacetylsilane-derivatized slides in discriminating single nucleotide polymorphisms is shown in FIG. 4. Four oligonucleotides differing in only the nucleotide at their (free) 3′-ends were arrayed. Were this array treated with a target complementary to one of these probes, no difference in hybridization at the four probe sites would be expected. However, when this array is treated with polymerase and fluoresceinated terminator, specific labeling of only the primer with perfect complementarity to the template is observed. Earlier work showed that catalysis by the polymerases used in APEX is very sensitive to mismatches at the 3′-end of the primer. They will not extend (and thereby label) primers with mismatches in the last five bases of the template. This is likely related to specific recognition of the last five base pairs of the primer-template complex by the polymerase, as recently demonstrated by crystallography (Kiefer et al, Nature 391:304 (1998)).

Most envisioned applications of APEX entail multiple templates in each reaction, and some methods (such as the molecular computation described by Connors et al, J. Am. Chem. Soc. in press) may involve multiple templates per priming site, for example as prepared by a binary combinatorial synthesis (Fodor et al, Science 251:767 (1991)). Examination of such situations was conducted using a 4-element grid in which four oligonucleotides are immobilized on film-coated slides. Two are spotted in each quadrant, as a 1:1 mixture of 1 mM oligonucleotides, creating a ‘two-bit’ experiment where each oligonucleotide can be represented by a bit, e.g.:

| Code | Oligonucleotides |
|---|---|
| 00 | 2/4 |
| 01 | 2/5 |
| 10 | 3/4 |
| 11 | 3/5 |

These spotting solutions were applied to A- and B-derivatized slides as shown:

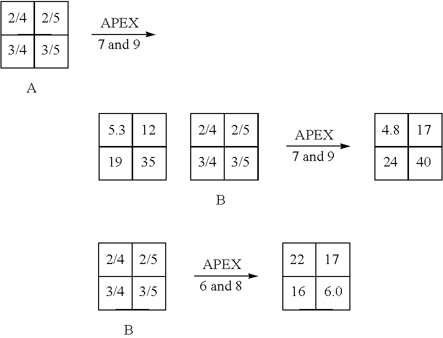

Two templates were used in APEX reaction mixtures such that one of the spotted quadrants would contain no complementary oligonucleotide, two of the quadrants would contain one complementary oligonucleotide, and one quadrant would contain two complementary oligonucleotides, for example, oligonucleotides 7 and 9, 6 and 8, 7 and 8, or 6 and 9 (see Table 1). Thus, after the APEX reaction, one quadrant should not contain a fluorescent spot, two quadrants should contain spots of intermediate fluorescence, and one quadrant should contain a spot that is highly fluorescent. The average S/N from 23 separate two-bit experiments each on A-derivatized slides with oligonucleotides 7 and 9 in the APEX reaction are shown above. The same two-bit experiment was performed 15 separate times on B-derivatized slides, giving the average S/N shown. A two-bit experiment with oligonucleotides 6 and 8 on B-derivatized slides gives the average S/N (18 spots each) shown above. The results are all comparable and excellent.

Another two-bit experiment was performed to examine the capability of APEX to discriminate against hybrids with a one base-pair mismatch one base from the 3′ end of the primer. Film-coated B slides were spotted with the oligonucleotide mixtures shown below. Two different APEX reaction mixtures were used for these mismatch experiments (see Table 1 for oligonucleotides). Controls utilized templates 15 and 17, which are perfectly complementary to 11 and 13, respectively. The experimental template solution contained oligonucleotides 7 and 17. As 17 and 13 can form a perfect hybrid, the right hand quadrants should be fluorescent. The lower left quadrant should not be fluorescent, as 7 and 11 would form a hybrid with a one base-pair T-T mismatch one base from the extension site. All other possible hybrids formed in this experiment will contain a mismatch at the 3′ end of the primer, which as shown by the foregoing experiments does not promote polymerase-dependent primer extension with fluorescein-ddATP. The S/N shown below reflect data from five different experiments.

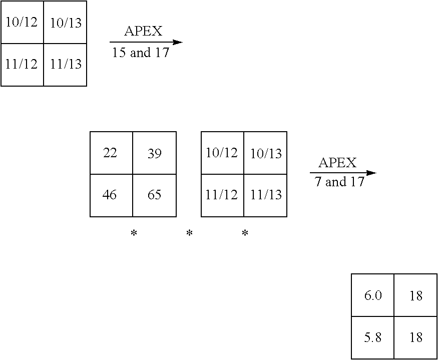

All documents cited above are hereby incorporated in their entirety by reference. One skilled in the art will appreciate from a reading of this disclosure that various changes in form and detail can be made without departing from the true scope of the invention.

1. A method of functionalizing a solid support comprising contacting said support with a bromoacetamidosilane of Formula I 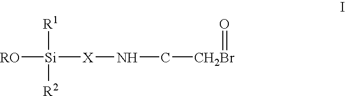
wherein:
R is an aryl or linear or branched alkyl,
R¹ and R² are, independently, a linear or branched alkyl, linear or branched alkoxy, aryl or phenoxy, and
X is a linker group, under conditions such that said bromoacetamidosilane reacts with groups present on said support.

2. The method according to claim 1 wherein
R is a linear or branched C₁-C₁₀ alkyl,
R¹ is a C₁-C₁₀ linear or branched alkyl and R² is a C₁-C₁₀ linear or branched alkyl, a C₁-C₁₀ linear or branched alkoxy or phenoxy, and
X is (CH₂)ₙ wherein n is 0 to 25.

3. The method according to claim 1 wherein said bromoacetamidosilane is N-(3-dimethylethoxysilylpropyl) bromoacetamide.

4. The method according to claim 1 wherein said solid support is a glass support.

5. A functionalized solid support produced according to the method of claim 1.

6. The functionalized support according to claim 5 wherein said functionalized support is a functionalized glass support.

7. A compound of the Formula I 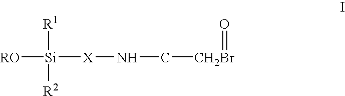
wherein:
R is an aryl or linear or branched alkyl,
R¹ and R² are, independently, a linear or branched alkyl, linear or branched alkoxy, aryl or phenoxy, and
X is a linker group.

8. The compund of claim 7 wherein
R is a linear or branched C₁-C₁₀ alkyl,
R¹ is a C₁-C₁₀ linear or branched alkyl and R² is a C₁-C₁₀ linear or branched alkyl, a C₁-C₁₀ linear or branched alkoxy or phenoxy, and
X is (CH₂)ₙ wherein n is 0 to 25.

9. A solid support silanized with a bromoacetamidosilane of Formula I: 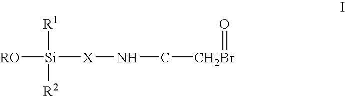
wherein:
R is an aryl or linear or branched alkyl,
R¹ and R² are, independently, a linear or branched alkyl, linear or branched alkoxy, aryl or phenoxy, and
X is a linker group.

10. The support according to claim 9 wherein said support is a glass support.

11. A method of immobilizing a phosphorothioate-terminated biopolymer on the support according to claim 9 comprising contacting said biopolymer with said support under conditions such that a bromoacetamide group present on said support reacts with said phosphorothioate.

12. The method according to claim 11 wherein said support is a glass support.

13. The method according to claim 11 wherein said biopolymer is a 5′ phosphorothioate oligonucleotide.

14. A composition comprising a phosphorothioate-terminated biopolymer and a bromoacetamidosilylated solid support wherein said phosphorothioate-terminated biopolymer is bound to said support via a bromoacetamide group.

15. The composition according to claim 14 wherein said biopolymer is a 5′ phosphorothioate oligonucleotide.

16. The composition according to claim 14 wherein said support is glass.

17. The composition according to claim 14 wherein said biopolymer bears a detectable label.

18. The composition according to claim 17 wherein said biopolymer is fluoresceinated.

19. A kit comprising the support according to claim 9 disposed within a container means.

20. A kit comprising the composition according to claim 14 disposed within a container means.


SEQUENCE LISTING (17 sequences)

SEQ ID NO: 1 - DNA, 20 bp, Artificial Sequence
  Description of Artificial Sequence Probe
cgcgaggtcgcacggctcag

SEQ ID NO: 2 - DNA, 25 bp, Artificial Sequence
  Description of Artificial Sequence Probe
cgcgaggtcgcacggctcagaaaaa

SEQ ID NO: 3 - DNA, 25 bp, Artificial Sequence
  Description of Artificial Sequence Probe
cgcgaggtcgcacggctcagaaaat

SEQ ID NO: 4 - DNA, 25 bp, Artificial Sequence
  Description of Artificial Sequence Probe
cgcgaggtcgcacggctcagaaaag

SEQ ID NO: 5 - DNA, 25 bp, Artificial Sequence
  Description of Artificial Sequence Probe
cgcgagctcgcacggctcagaaaac

SEQ ID NO: 6 - DNA, 29 bp, Artificial Sequence
  Description of Artificial Sequence Probe
tttttttttctgagccgtgcgacctcgcg

SEQ ID NO: 7 - DNA, 29 bp, Artificial Sequence
  Description of Artificial Sequence Probe
ttttattttctgagccgtgcgacctcgcg

SEQ ID NO: 8 - DNA, 29 bp, Artificial Sequence
  Description of Artificial Sequence Probe
ttttcttttctgagccgtgcgacctcgcg

SEQ ID NO: 9 - DNA, 29 bp, Artificial Sequence
  Description of Artificial Sequence Probe
ttttgttttctgagccgtgcgacctcgcg

SEQ ID NO: 10 - DNA, 25 bp, Artificial Sequence
  Description of Artificial Sequence Probe
cgcgaggtcgcacggctcagaaata

SEQ ID NO: 11 - DNA, 25 bp, Artificial Sequence
  Description of Artificial Sequence Probe
cgcgaggtcgcacggctcagaaatt

SEQ ID NO: 12 - DNA, 25 bp, Artificial Sequence
  Description of Artificial Sequence Probe
cgcgaggtcgcacggctcagaaatg

SEQ ID NO: 13 - DNA, 25 bp, Artificial Sequence
  Description of Artificial Sequence Probe
cgcgaggtcgcacggctcagaaatc

SEQ ID NO: 14 - DNA, 29 bp, Artificial Sequence
  Description of Artificial Sequence Probe
tttttatttctgagccgtgcgacctcgcg

SEQ ID NO: 15 - DNA, 29 bp, Artificial Sequence
  Description of Artificial Sequence Probe
ttttaatttctgagccgtgcgacctcgcg

SEQ ID NO: 16 - DNA, 29 bp, Artificial Sequence
  Description of Artificial Sequence Probe
ttttcatttctgagccgtgcgacctcgcg

SEQ ID NO: 17 - DNA, 29 bp, Artificial Sequence
  Description of Artificial Sequence Probe
ttttgatttctgagccgtgcgacctcgcg